(19) United States
(12) Patent Application Publication
(10) Pub. No.: US 2010/0209911 A1
(43) Pub. Date: Aug. 19, 2010

(54) Microsatellite maker combination and method for identifying Lanyu pig breed

(75) Inventors: Yu-Ten Ju, Taipei City (TW); Yan-Nian Jiang, Taipei City (TW); Hsien-Ping Chu, Taitung County (TW); Kuen-Jaw Chen, Taitung County (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei City (TW)

(21) Appl. No.: 12/461,527

(22) Filed: Aug. 14, 2009

(30) Foreign Application Priority Data
Feb. 16, 2009 (TW) 98104881

(51) Int. Cl. C12Q 1/68; C07H 21/04

(52) U.S. Cl. 435/6; 536/23.1

(57) ABSTRACT

The present invention provides a microsatellite marker combination for identifying Lanyu pig breed, and the identification method thereof. The identification method comprises the following steps: (a) providing a genomic DNA sample obtained from a pig; (b) identifying the polymorphism of microsatellite markers of said genomic DNA sample; and (c) analyzing the results obtained from step (b) to determine the phylogenetic relationship between said pig and Lanyu pig.

### BACKGROUND OF THE INVENTION

1. Field of the Invention

The present invention relates to a microsatellite marker combination for identifying Lanyu pig breed and the identification method thereof.

2. Description of the Related Art

Lanyu pig is an indigenous miniature pig breed of Taiwan. This small-eared pig breed originates from Lanyu Islet that is off the southeastern coast of Taiwan main island. It has a long head, short and strong limbs, small and up-straight ears, a slightly concave back, black coarse hair and 14 pairs of ribs. The average weight of 22 week-old male pigs is 17.95±6.53 kg, and that of 22 week-old female pigs is 16.21±3.29 kg. The number of liveborn piglets is about 5.6 (see Chang et al., 1998b). In addition, the weight of an adult Lanyu pig is approximately 75 kg. After selection and breeding in Taitung Animal Propagation Station (TAPS), a propagation station of Taiwan Livestock Research Institute (TLRI), Peinan Lanyu pig with pure white fur is selected and registered in 2002 and Spotty Lanyu pig with black and white spotty fur is selected and registered in 2003.

In 1975, Department of Animal Science of National Taiwan University (NTU), now known as Department of Animal Science and Technology, introduced one male small-eared pig and three female small-eared pigs from Lanyu. Later, TAPS introduced other four male small-eared pig and sixteen female small-eared pigs from Lanyu in 1980. After that, in order to set up a miniature pig based population for medical research, a Lanyu pig population comprising 15 male and 45 female pigs was kept by natural mating. In 1987, the conservation population of Lanyu pigs introduced from Lanyu to Taiwan was incorporated as a national conservation population by Council of Agriculture of Taiwan (see Chang 1998b). These pigs are now under ex situ conservation in NTU Experimental Farm and TAPS.

The genetic constitution of Lanyu pig is unique. According to phylogenetic study from mitochondrial D-loop sequences analysis, the genetic distances between Lanyu pig and other European and Asian pig breeds (miniature pigs are included) are quite far. Therefore, using Lanyu pig to imitate human body in medical experiments will be different from other miniature pigs. In other words, Lanyu pig has a potential to be a unique animal model for medical experiments, which cannot be substituted by other experimental pigs. If Lanyu pig becomes a general laboratory animal, the genetic identification and quality control of its genetic constitution will be very important.

### SUMMARY OF THE INVENTION

In recent 50 years, many polymorphism-associated genetic markers have been applied to study the genetic relationship between species and breed identification, including identifications and studies of allozyme, DNA sequence polymorphism and DNA repeat sequence polymorphism. The present invention provides a method for breed identification by use of DNA repeat sequence polymorphism, and a microsatellite marker combination for the breed identification.

Many tandemly-repeated sequences are scattered in eukaryotic chromosome. These sequences are constituted by repeats of a specific unit of sequence, and they can be categorized according to the length of the repeat fragment into satellite DNA, which has a length up to 5 Mb; minisatellite DNA, which having a length of approximately 0.5-3 kb that is usually constituted by a tandemly-repeated sequence of 14 to 100 nucleosides; and microsatellite DNA, which having a length of 20-200 bp that is usually constituted by a tandemly-repeated sequence of 1 to 6 nucleotides. Since many tandemly-repeated sequences have a high level of polymorphism, and they are dispersed in specific regions of chromosomes, these tandemly-repeated sequences are commonly used as genetic molecular markers of species for gene mapping, phylogenetic identification, and genetic relationship studies.

Microsatellite DNA has a high mutation rate, consequently, it has a high level of polymorphism between individuals, and widely used as a molecular marker for phylogenetic identification between species or paternity identification within a species. The method of the present invention amplified one microsatellite marker in samples obtained from different individuals with fluorescence-labeled primers, and then the amplified fragments were subjected to capillary electrophoresis; after that, the genetic distance between individuals were calculated according to the electrophoresis result, and the phylogenetic tree was constructed to identify the genetic relationship between individuals or between species.

Therefore, the present invention provides a microsatellite marker combination for identifying Lanyu pig breed, comprising SW024, SW72, SW122, SW857, SW911, SW951, IGF1, S0002, S0005, S0068, S0155, S0215, S0218, S0225, S0226, S0227, S0228, S0355 and S0386.

The present invention also provides a method for identifying Lanyu pig breed, comprising the following steps: (a) providing a genomic DNA sample obtained from a pig; (b) identifying the polymorphism of microsatellite markers of said genomic DNA sample, wherein said microsatellite markers comprising SW024, SW72, SW122, SW857, SW911, SW951, IGF1, S0002, S0005, S0068, S0155, S0215, S0218, S0225, S0226, S0227, S0228, S0355 and S0386; and (c) analyzing the results obtained from step (b) to determine the phylogenetic relationship between said pigs and Lanyu pig.

In preferred embodiments of the present invention, the polymorphism of said microsatellite markers is identified by polymerase chain reaction and capillary electrophoresis.

In preferred embodiments of the present invention, the phylogenetic relationship between said pig and Lanyu pig is determined by constructing a phylogenetic tree.

In preferred embodiments of the present invention, the polymorphism of said microsatellite markers comprises one or more private alleles selected from: the repeat fragment of SW024 having a length of 118 bp; the repeat fragment of SW911 having a length of 163 bp; the repeat fragment of SW951 having a length of 136 bp; the repeat fragment of S0002 having a length of 174 bp; the repeat fragment of S0068 having a length of 244 bp; the repeat fragment of S0155 having a length of 166 bp; the repeat fragment of S0218 having a length of 188 bp; the repeat fragment of S0225 having a length of 174 bp; the repeat fragment of S0226 having a length of 176 bp; the repeat fragment of S0227 having a length of 238 bp; the repeat fragment of S0227 having a length of 258 bp; the repeat fragment of S0228 having a length of 227 bp; the repeat fragment of S0355 having a length of 272 bp; or the repeat fragment of S0355 having a length of 276 bp.

### BRIEF DESCRIPTION OF THE DRAWINGS

FIG. 1 is the phylogenetic tree constructed in accordance with the genetic distances of −ln (proportion of shared alleles) between 242 individual pigs, in which the genetic distances are calculated according to the polymorphism of the 19 microsatellite makers of the present invention. FIG. 1 shows that each of the seven pig breeds used in the present invention forms an individual branch. The individual pigs mingled in the branch that is different with its own breed are labeled in red.

### DETAILED DESCRIPTION OF THE PREFERRED EMBODIMENTS

In the present invention, 19 pairs of fluorescence-labeled primers of microsatellite makers are used in the polymerase chain reaction (PCR) for genomic DNA samples from 7 pig breeds (242 individual pigs in total). After that, the amplified repeat fragments of microsatellite markers are subjected to capillary electrophoresis to determine the length of repeated sequence and the polymorphism of microsatellite markers of each individual pig. After that, the polymorphism data is calculated by software such like MSA, Structure, CERVUS etc. to obtain genotype frequency, genetic distance, phylogenetic tree, heterozygosity of each gene locus, private alleles and the like, which can be used for comparing the phylogenetic relationship of these individual pigs.

Pig Breeds and their Sources

The samples of Lanyu pig breed used in the present invention were collected from 5 Lanyu pigs in NTU Experimental Farm affiliated to the College of Bio-Resources and Agriculture of National Taiwan University and 39 Lanyu pigs in Taitung Animal Propagation Station (44 Lanyu pigs in total). The samples of Taoyuan pig breed were collected from 6 Taoyuan pigs in Kaohsiung Animal Propagation Station and 30 Taoyuan pigs in Taiwan Livestock Research Institute (TLRI) (36 Taoyuan pigs in total). The samples of Meishan pig breed were collected from 7 Meishan pigs in Kaohsiung Animal Propagation Station and 30 Meishan pigs in TLRI (37 Meishan pigs in total). Furthermore, the samples of 32 Landrace pigs, 31 Yorkshire pigs, 30 Berkshire pigs and 32 Duroc pigs were collected in TLRI. These samples were collected from 7 pig breeds, 242 pigs in total, as shown in Table 1.

TABLE 1

| Pig breed | Source | Number |
|---|---|---|
| Lanyu | Taitung Animal Propagation Station | 39 |
| | NTU Experimental Farm | 5 |
| Taoyuan | Kaohsiung Animal Propagation Station | 6 |
| | TLRI | 30 |
| Meishan | Kaohsiung Animal Propagation Station | 7 |
| | TLRI | 30 |
| Landrace | TLRI | 32 |
| Yorkshire | TLRI | 31 |
| Duroc | TLRI | 32 |
| Berkshire | TLRI | 30 |

### Collection of Blood Samples

The pig blood samples of the present invention were collected at the jugular bulb of the 242 pigs listed in Table 1. The wall of the 20 mL syringes were adequately rinsed by 1 mL of EDTA (0.5M, pH 8.0). 30 mL of blood was collected from each pig, and the blood sample was filled in a collecting tube comprising EDTA (BD Vacutainer™ K3EDTA, USA) and placed in an ice bucket immediately.

### Preparation of Genomic DNA

10 mL blood, taken from each of the above-mentioned blood samples, was used to extract the genomic DNA by QIAamp DNA Blood Maxi Kit (QIAGEN, USA) in accordance with the instruction appendixed in the kit. The obtained genomic DNA was loaded in 0.6% agar gel and separated in 0.5×TBE buffer by 100V electrophoresis. After that, the quality of genomic DNA was confirmed via its electrophoresis band pattern, and the genomic DNA was stored in a −20° C. freezer.

### Primer Design of Microsatellite Makers

19 microsatellite makers scattered in 15 pairs of chromosomes of pigs' total 19 pairs of genomes were selected, comprising SW024, SW72, SW122, SW857, SW911, SW951, IGF1, S0002, S0005, S0068, S0155, S0215, S0218, S0225, S0226, S0227, S0228, S0355 and S0386. The primers synthesized by MWG Biotech (England) were used to amplify sequence fragments with different lengths by PCR, thereby analyzing the polymorphism of each microsatellite marker. The 5′ end of these primers was fluorescence labeled. The sequences of the primers were listed in Table 2.

TABLE 2
Primers of microsatellite markers

| Micro- | Chromo- | Repeat | | | Annealing | |
|---|---|---|---|---|---|---|
| satellite | somal | fragment | Forward (5′) primer sequence | | temp | Fluores- |
| marker | location | length (bp) | Reverse (3′) primer sequence | SEQ ID No. | (° C.) | cence |
| SW024 | 17q | 96-120 | 5′-CTTTGGGTGGAGTGCGTGC- 3′ | (SEQ ID NO: 1) | 58 | HEX |
| | | | 5′-ATCCAAATGCTGCAAGCG-3′ | (SEQ ID NO: 2) | | |
| SW72 | 3p | 42-162 | 5′-TGAGAGGTCAGTTACAGAAGACC-3′ | (SEQ ID NO: 3) | 58 | HEX |
| | | | 5′-GATCCTCCTCCAAATCCCAT-3′ | (SEQ ID NO: 4) | | |
| SW122 | 6q | 116-138 | 5′-TTGTCTTTTTATTTTGCTTTTGG-3′ | (SEQ ID NO: 5) | 58 | HEX |
| | | | 5′-CAAAAAAGGCAAAAGATTGACA-3′ | (SEQ ID NO: 6) | | |
| SW857 | 14q | 96-114 | 5′-AGAAATTAGTGCCTCAAATTGG-3′ | (SEQ ID NO: 7) | 58 | FAM |
| | | | 5′-AAACCATTAAGTCCCTAGCAAA-3′ | (SEQ ID NO: 8) | | |
| SW911 | 9p | 157-173 | 5′-CTCAGTTCTTTGGGACTGAACC-3′ | (SEQ ID NO: 9) | 58 | FAM |
| | | | 5′-CATCTGTGGAAAAAAAAAGCC-3′ | (SEQ ID NO: 10) | | |
| SW951 | 10q | 120-136 | 5′-TTTCACAACTCVGGCACCAG-3′ | (SEQ ID NO: 11) | 58 | HEX |
| | | | 5′-GATCCTGCCCAAATGGAC-3′ | (SEQ ID NO: 12) | | |
| IGF1 | 5q | 194-250 | 5′-GCTTGGATGGACCATGTTG-3′ | (SEQ ID NO: 13) | 58 | TAMRA |
| | | | 5′-CATATTTTTCTGCATAACTTGAACCT-3′ | (SEQ ID NO: 14) | | |
| S0002 | 3q | 174-220 | 5′-GAAGCCCAAAGAGACAACTGC-3′ | (SEQ ID NO: 15) | 62 | FAM |
| | | | 5′-GTTCTTTACCCACTGAGCCA-3′ | (SEQ ID NO: 16) | | |
| S0005 | 5q | 206-278 | 5′-TCCTTCCCTCCTGGTAACTA-3′ | (SEQ ID NO: 17) | 58 | FAM |
| | | | 5′-GCACTTCCTGATTCTGGGTA-3′ | (SEQ ID NO: 18) | | |
| S0068 | 13q | 190-262 | 5′-AGTGGTCTCTCTCCCTCTTGCT-3′ | (SEQ ID NO: 19) | 58 | TAMRA |
| | | | 5′-CCTTCAACCTTTGCGCAAGAAC-3′ | (SEQ ID NO: 20) | | |
| S0155 | 1q | 148-170 | 5′-TGTTCTCTGTTTCTCCTCTGTTTG-3′ | (SEQ ID NO: 21) | 55 | FAM |
| | | | 5′-AAAGTGGAAAGAGTCAATGGCTAT-3′ | (SEQ ID NO: 22) | | |
| S0215 | 13q | 138-170 | 5′-TAGGCTCAGACCCTGCTGCAT-3′ | (SEQ ID NO: 23) | 55 | HEX |
| | | | 5′-TGGGAGGCTGAAGGATTGGGT-3′ | (SEQ ID NO: 24) | | |
| S0218 | x | 158-188 | 5′-GTGTAGGCTGGCGGTTGT-3′ | (SEQ ID NO: 25) | 55 | FAM |
| | | | 5′-CCCTGAAACCTAAAGCAAAG-3′ | (SEQ ID NO: 26) | | |
| S0225 | 8q | 170-194 | 5′-GCTAATGCCAGAGAAATGCAGA-3′ | (SEQ ID NO: 27) | 55 | FAM |
| | | | 5′-CAGGTGGAAAGAATGGAATGAA-3′ | (SEQ ID NO: 28) | | |
| S0226 | 2q | 176-214 | 5′-GCACTTTTAACTTTCATGATACTCC-3′ | (SEQ ID NO: 29) | 55 | FAM |
| | | | 5′-GGTTAAACTTTTNCCCCAATACA-3′ | (SEQ ID NO: 30) | | |
| S0227 | 4p | 228-258 | 5′-GATCCATTTATAATTTTAGCACAAAGT-3′ | (SEQ ID NO: 31) | 58 | TAMRA |
| | | | 5′-GCATGGTGTGATGCTATGTCAAGC-3′ | (SEQ ID NO: 32) | | |
| S0228 | 6q | 221-243 | 5′-GGCATAGGCTGGCAGCAACA-3′ | (SEQ ID NO: 33) | 58 | TAMRA |
| | | | 5′-AGCCCACCTCATCTTATCTACACT-3′ | (SEQ ID NO: 34) | | |
| S0355 | 15q | 236-276 | 5′-TCTGGCTCCTACACTCCTTCTTGATG-3′ | (SEQ ID NO: 35) | 58 | TAMRA |
| | | | 5′-TTGGGTGGGTGCTGAAAAATAGGA-3′ | (SEQ ID NO: 36) | | |
| S0386 | 11q | 156-172 | 5′-TCCTGGGTCTTATTTTCTA-3′ | (SEQ ID NO: 37) | 48 | FAM |
| | | | 5′-TTTTTATCTCCAACAGTAT-3′ | (SEQ ID NO: 38) | | |

### Polymerase Chain Reaction

In the present invention, polymerase chain reaction (PCR) was processed by using Taq DNA polymerase (Amersham Biosciences, USA) and PTC-200 Programmable Thermal Controller (MJ Research Inc., USA). First, 11.105 μL sterile ddH₂O, 1.5 μL Taq DNA polymerase 10× buffer (500 mM KCl, 15 mM MgCl₂ and 100 mM Tris-HCl), 0.45 μL one pair of the microsatellite primers (4.5 pmole), 0.375 μL 8 mM dNTP and 0.12 μL Taq DNA polymerase (5 U/4 μL, comprising 50 mM Tris-HCl, 0.1 mM EDTA and 5 mM DTT) were added into a sterile 1.5 mL microcentrifuge tube, vortex mixed well. After that, 14 μL it of the mixture was added into each well of a 96-well PCR reacting tray (Sorenson Bioscience, USA) and then 1 μL of genomic DNA (50 ng/μL) was added, so the total PCR reaction volume is 15 μL. The tray was patted to mix said mixture and genomic DNA well, and then centrifuged at 4° C. for 2 minutes at 2000 rpm to deposit the reaction mixture to the lower part of the well, thereby facilitating the PCR reaction. Finally, the reaction tray was placed in PTC-200 when the upper and lower heaters were heated to 95° C. for processing PCR reaction. The conditions of PCR reaction are as follows:

Step 1: 95° C., 5 minutes 1 cycle;

Step 2: 95° C., 30 seconds;
48-62° C. (see the annealing temperature list in Table 2), 30 seconds;
72° C., 45 seconds;
Step 2 repeated for 37 cycles;

Step 3: 72° C., 7 minutes 1 cycle.

### Analysis of Polymorphism of Microsatellite Markers

3 μL product of the above-mentioned PCR reaction was taken out for capillary electrophoresis. After that, MEGABACE 1000 sequencer (Amershan Biosciences, USA) and software Genetic-profiler Version 2.2 (Amershan Biosciences, USA) were used for DNA sequencing and analyzing the length polymorphism of the amplified DNA fragment of all microsatellite markers, i.e. the length polymorphism caused by the repeating number of a tandem repeat sequence on two allele loci. In addition, the fluorescence label ET-400 (Amershan Biosciences, USA) was used as a calibration standard for allele length.

### Construction of Neighbor Joining Tree (NJ Tree)

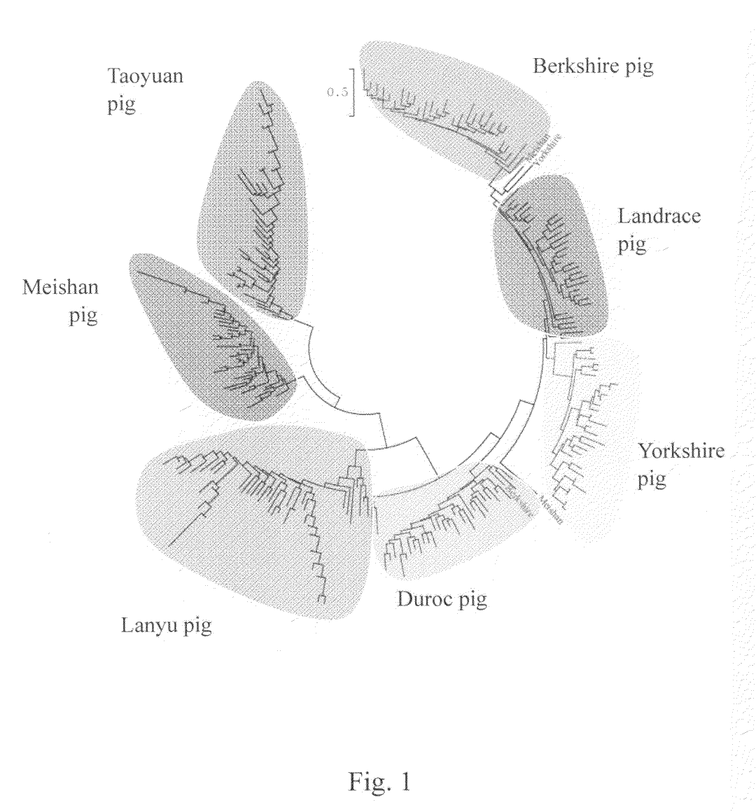

The genetic distances of −ln (proportion of shared alleles) between the 242 individual pigs were calculated by MSA software in accordance with the polymorphism of the 19 microsatellite markers. Afterwards, a neighbor joining tree was constructed by the software MEGA3 (see Kumar et al., 2000). The result showed that each of the seven pig breeds formed an individual branch, as shown in FIG. 1.

### Comparison of Alleles

Additionally, alleles of Lanyu pigs, Taoyuan pigs, Meishan pigs, Landrace pigs, Yorkshire pigs, Duroc pigs and Berkshire pigs were further compared according to the polymorphism of the above-mentioned 19 microsatellite markers. The results showed that Lanyu pigs had 14 private alleles scattered on 12 loci.

Table 3 shows 44 conserved Lanyu pigs used in the experiments of the present invention and the comparison of their alleles. In this Table, the term “effective allele number” means that the allele number of the microsatellite marker of the present invention is higher enough to be used for population genetic polymorphism analysis; and the term “private allele” means that the allele of the microsatellite locus of the present invention is unique in a specific population or a specific species, not shown in other population or species.

TABLE 3
Private alleles located on 19 microsatellite loci of 44
conserved Lanyu pigs in Taiwan main island

| Microsatellite | | Effective allele | Private allele number |
|---|---|---|---|
| Marker | Allele number | number | (fragment length) |
| SW024 | 3 | 1.45 | 1 (118 bp) |
| SW72 | 5 | 2.48 | |
| SW122 | 4 | 3.06 | |
| SW857 | 3 | 1.90 | |
| SW911 | 3 | 2.12 | 1 (163 bp) |
| SW951 | 5 | 2.75 | 1 (136 bp) |
| IGF1 | 3 | 2.38 | |
| S0002 | 5 | 3.33 | 1 (174 bp) |
| S0005 | 2 | 1.92 | |
| S0068 | 4 | 3.07 | 1 (244 bp) |
| S0155 | 3 | 2.65 | 1 (166 bp) |
| S0215 | 2 | 1.38 | |
| S0218 | 4 | 3.22 | 1 (188 bp) |
| S0225 | 3 | 2.17 | 1 (174 bp) |
| S0226 | 6 | 2.41 | 1 (176 bp) |
| S0227 | 4 | 1.45 | 2 (238, 258 bp) |
| S0228 | 3 | 2.24 | 1 (227 bp) |
| S0355 | 6 | 2.54 | 2 (272, 276 bp) |
| S0386 | 3 | 2.92 | |
| Mean | 3.74 | 2.39 | 0.73 |

In all 242 individual pigs of the present invention, 4 individual pigs were divided into a branch different with its own breed. They are 2 Meishan pigs, 1 Yorkshire pig and 1 Berkshire pig. This result shows that the gene of these 4 pigs might be transgressed by other pig breed's gene. However, the identification method of the present invention can exactly separate the Lanyu pig breed and other pig breeds. Furthermore, the 39 Lanyu pigs of Taitung Animal Propagation Station and the 5 Lanyu pigs from NTU Experimental Farm are divided into 2 small branches. This may be because these two subgroups were introduced at different times and bred separately for years, so their genetic frequencies are different, and there are larger genetic distances between these individual Lanyu pigs. In this analysis, the correct rate of correctly dividing an individual pig into its breed branch is 98.4%. In other words, the neighbor joining tree analysis according the −ln (proportion of shared allele) distances between the 242 individual pigs has high identification ability among these pig breeds.

Lanyu pig now has been developed to be a medium to large sized animal model, and they were adopted as an animal model for biomedical researches in hospitals and research centers. The researchers or animal model breeding companies are going to cross Lanyu pig and other miniature pigs to breed new synthesized pig breed in the future, so the possibility of Lanyu pig crossing with other pigs will be greater and greater. If Lanyu pig becomes an animal model for international exchanges, the genetic monitoring and identification will be a very important issue. The microsatellite markers and private alleles of the present invention can be used not only as a genetic identification label in the traceability system, but also as a genetic monitoring marker.

What is claimed is:

1. A microsatellite marker combination for identifying Lanyu pig breed, comprising SW024, SW72, SW122, SW857, SW911, SW951, IGF1, S0002, S0005, S0068, S0155, S0215, S0218, S0225, S0226, S0227, S0228, S0355 and S0386.

2. A method for identifying Lanyu pig breed, comprising the following steps:
(a) providing a genomic DNA sample obtained from a pig;
(b) identifying the polymorphism of microsatellite markers of said genomic DNA sample, wherein said microsatellite markers comprising SW024, SW72, SW122, SW857, SW911, SW951, IGF1, S0002, S0005, S0068, S0155, S0215, S0218, S0225, S0226, S0227, S0228, S0355 and S0386;
(c) analyzing the results obtained from step (b) to determine the phylogenetic relationship between said pig and Lanyu pig.

3. The method according to claim 2, wherein the polymorphism of said microsatellite markers is identified by polymerase chain reaction.

4. The method according to claim 2, wherein the phylogenetic relationship between said pig and Lanyu pig is determined by constructing a phylogenetic tree.

5. The method according to claim 2, wherein the polymorphism of said microsatellite markers comprises one or more private alleles selected from:
the repeat fragment of SW024 having a length of 118 bp;
the repeat fragment of SW911 having a length of 163 bp;
the repeat fragment of SW951 having a length of 136 bp;
the repeat fragment of S0002 having a length of 174 bp;
the repeat fragment of S0068 having a length of 244 bp;
the repeat fragment of S0155 having a length of 166 bp;
the repeat fragment of S0218 having a length of 188 bp;
the repeat fragment of S0225 having a length of 174 bp;
the repeat fragment of S0226 having a length of 176 bp;
the repeat fragment of S0227 having a length of 238 bp;
the repeat fragment of S0227 having a length of 258 bp;
the repeat fragment of S0228 having a length of 227 bp;
the repeat fragment of S0355 having a length of 272 bp; or
the repeat fragment of S0355 having a length of 276 bp.


SEQUENCE LISTING (38 sequences)

SEQ ID NO: 1 - DNA, 19 bp, Artificial
  primer
ctttgggtggagtgcgtgc

SEQ ID NO: 2 - DNA, 18 bp, Artificial
  primer
atccaaatgctgcaagcg

SEQ ID NO: 3 - DNA, 23 bp, Artificial
  primer
tgagaggtcagttacagaagacc

SEQ ID NO: 4 - DNA, 20 bp, Artificial
  primer
gatcctcctccaaatcccat

SEQ ID NO: 5 - DNA, 23 bp, Artificial
  primer
ttgtctttttattttgcttttgg

SEQ ID NO: 6 - DNA, 22 bp, Artificial
  primer
caaaaaaggcaaaagattgaca

SEQ ID NO: 7 - DNA, 22 bp, Artificial
  primer
agaaattagtgcctcaaattgg

SEQ ID NO: 8 - DNA, 22 bp, Artificial
  primer
aaaccattaagtccctagcaaa

SEQ ID NO: 9 - DNA, 22 bp, Artificial
  primer
ctcagttctttgggactgaacc

SEQ ID NO: 10 - DNA, 21 bp, Artificial
  primer
catctgtggaaaaaaaaagcc

SEQ ID NO: 11 - DNA, 20 bp, Artificial
  primer
tttcacaactcvggcaccag

SEQ ID NO: 12 - DNA, 18 bp, Artificial
  primer
gatcctgcccaaatggac

SEQ ID NO: 13 - DNA, 19 bp, Artificial
  primer
gcttggatggaccatgttg

SEQ ID NO: 14 - DNA, 26 bp, Artificial
  primer
catatttttctgcataacttgaacct

SEQ ID NO: 15 - DNA, 21 bp, Artificial
  primer
gaagcccaaagagacaactgc

SEQ ID NO: 16 - DNA, 20 bp, Artificial
  primer
gttctttacccactgagcca

SEQ ID NO: 17 - DNA, 20 bp, Artificial
  primer
tccttccctcctggtaacta

SEQ ID NO: 18 - DNA, 20 bp, Artificial
  primer
gcacttcctgattctgggta

SEQ ID NO: 19 - DNA, 22 bp, Artificial
  primer
agtggtctctctccctcttgct

SEQ ID NO: 20 - DNA, 22 bp, Artificial
  primer
ccttcaacctttgcgcaagaac

SEQ ID NO: 21 - DNA, 24 bp, Artificial
  primer
tgttctctgtttctcctctgtttg

SEQ ID NO: 22 - DNA, 24 bp, Artificial
  primer
aaagtggaaagagtcaatggctat

SEQ ID NO: 23 - DNA, 21 bp, Artificial
  primer
taggctcagaccctgctgcat

SEQ ID NO: 24 - DNA, 21 bp, Artificial
  primer
tgggaggctgaaggattgggt

SEQ ID NO: 25 - DNA, 18 bp, Artificial
  primer
gtgtaggctggcggttgt

SEQ ID NO: 26 - DNA, 20 bp, Artificial
  primer
ccctgaaacctaaagcaaag

SEQ ID NO: 27 - DNA, 22 bp, Artificial
  primer
gctaatgccagagaaatgcaga

SEQ ID NO: 28 - DNA, 22 bp, Artificial
  primer
caggtggaaagaatggaatgaa

SEQ ID NO: 29 - DNA, 25 bp, Artificial
  primer
gcacttttaactttcatgatactcc

SEQ ID NO: 30 - DNA, 23 bp, Artificial
  primer
ggttaaacttttnccccaataca

SEQ ID NO: 31 - DNA, 27 bp, Artificial
  primer
gatccatttataattttagcacaaagt

SEQ ID NO: 32 - DNA, 24 bp, Artificial
  primer
gcatggtgtgatgctatgtcaagc

SEQ ID NO: 33 - DNA, 20 bp, Artificial
  primer
ggcataggctggcagcaaca

SEQ ID NO: 34 - DNA, 24 bp, Artificial
  primer
agcccacctcatcttatctacact

SEQ ID NO: 35 - DNA, 26 bp, Artificial
  primer
tctggctcctacactccttcttgatg

SEQ ID NO: 36 - DNA, 24 bp, Artificial
  primer
ttgggtgggtgctgaaaaatagga

SEQ ID NO: 37 - DNA, 19 bp, Artificial
  primer
tcctgggtcttattttcta

SEQ ID NO: 38 - DNA, 19 bp, Artificial
  primer
tttttatctccaacagtat